(19) United States
(12) Patent Application Publication
(10) Pub. No.: US 2015/0088152 A1
(43) Pub. Date: Mar. 26, 2015

(54) ELONGATED MEMBER FOR MEDICAL USE

(71) Applicant: TERUMO KABUSHIKI KAISHA, TOKYO (JP)

(72) Inventor: TOMONORI HATTA, KANAGAWA (JP)

(21) Appl. No.: 14/495,524

(22) Filed: Sep. 24, 2014

(30) Foreign Application Priority Data
Sep. 25, 2013 (JP) 2013-197939

(51) Int. Cl. A61B 17/00

(52) U.S. Cl. CPC A61B 17/00234
USPC 606/108

(57) ABSTRACT

An elongated member for medical use that is used to perform a procedure in a pulmonary airway. The elongated member suitable for a procedure in a pulmonary airway comprises plural members connected to each other that have different lumen diameters, outer diameters, and physical properties, and which further includes an operating member that has an elongated shape and is connected to at least part of the first member, and which manipulates the curving the first member through push/pull operation.

### CROSS REFERENCE TO RELATED APPLICATIONS

This application claims priority to Japanese National Application No. 2013-197939, filed on Sep. 25, 2013. The entire contents of which is incorporated herein by reference.

### BACKGROUND

The present invention relates to an elongated member for medical use, particularly for performing a procedure in a pulmonary airway.

An elongated member for medical use is known that is used in performing various kinds of procedures in a pulmonary airway of a living body, such as diagnosis of the disease state of a patient, treatment for a lesion, and delivery of various kinds of media into a biological organ.

For example, the elongated member is used to assist the movement of a medical device for achieving a diagnostic image such as an endoscopic image when the medical device is moved to a lesion in some cases. Furthermore, it is used to guide a medical instrument, such as a biopsy instrument, to a lesion in a manipulation of, for example, cutting a biological tissue. The elongated member is often formed integrally with a catheter device and used for delivery of various kinds of medications into a living body and for suction of a body fluid or other substances in a living body.

The elongated member for medical use is widely used for procedures of the respective organs of living bodies. Furthermore, the elongated member is so designed that its specifications such as shape, dimensions, and physical properties are suitable for the biological organ as the application target. For example, in an elongated member used to perform various kinds of procedures in a pulmonary airway of a living body, the distal side is formed with a smaller diameter in order to enhance the reachability of the elongated member to the peripheral side of bronchi. Additionally, the distal side is so formed as to be capable of curving in association with operation at the hand side so that the elongated member can be moved along the pulmonary airway having bifurcations and a tortuous shape. Meanwhile, with regard to the delivery of various types of procedure instruments, medications, and the like via the inner cavity (lumen) of the elongated member, the proximal side of the elongated member has a larger lumen diameter. In addition, with regard to the operability of the elongated member in the pulmonary airway, it is more preferable that the distal side be endowed with flexibility and the proximal side be endowed with pushability (i.e., transmissibility of a pushing force).

For example, Japanese Patent Laid-Open No. 2012-196389 discloses an elongated member for medical use that is given a tapered shape in which the lumen diameter and the outer diameter become smaller toward the distal side and is so formed such that the physical properties (hardness and rigidity) of the respective parts are different in the axial line direction. Specifically, as a method for manufacturing the elongated member, a method is employed in which plural outer layer members having different physical properties are disposed as covering members at different positions, in the axial line direction, on a base member composed of a single material. By this method, realization of the elongated member in which the hardness becomes lower toward the distal side is enabled.

### SUMMARY OF THE DISCLOSURE

The related art described in Japanese Patent Laid-Open No 2012-196389 provides an elongated member that has the desired lumen diameter and outer diameter and in which flexibility is ensured on the distal side. However, because the elongated member described in Japanese Patent Laid-Open No 2012-196389 is formed by using the base member composed of a single material, the elongated member has at least physical properties according to the material of this base member. For example, when forming the elongated member in such a manner that the physical properties of the distal side of the elongated member are different from those of the proximal side, specifically in the case of attempting to facilitate more flexible deformation of the distal side and endow the proximal side with higher pushability, it is difficult to endow the elongated member with desired physical properties by merely depositing the outer layer members as covering members because the physical properties of the elongated member depend on and/or effect the physical properties of the base member.

As a countermeasure against the above-described problem, provision of an elongated member suitable for a procedure in a pulmonary airway would be enabled by manufacturing the elongated member by connecting plural members different from each other in lumen diameter, outer diameter, and physical properties. However, problems occur when the elongated member is formed by connecting plural members.

As described above, in the elongated member used in a pulmonary airway, the distal side is so formed as to be capable of curving so that the operability can be enhanced. For this purpose, the elongated member is provided with a predetermined operating member configured to operate the curving action of the distal side. The operating member generally used is so formed as to curve the distal side in association with push/pull operation when the push/pull operation is carried out on the hand side. This operating member is extended toward the side of a hand operating part via the distal side and proximal side of the elongated member. Therefore, at the connection parts across which the plural members are connected, the operating member is so disposed as to straddle the respective members.

In the manufacturing of the elongated member, the operating member could be easily extended from the distal side toward the proximal side and the manufacturing process would be facilitated by making the operating member pass through the outer surface side of the respective members to be extended along the axial direction or pass through the inner surface side of the respective members to be extended along the axial direction. However, when using members having different lumen diameter and outer diameters, the operating member is compelled to be so disposed as to be inclined or bent radially outward at the connection part as the joint across which both members are connected. If the operating member is disposed in this manner, the smooth push/pull operation of the operating member along the axial direction is hindered. Therefore, the resulting performance of the curving action is lowered. In addition, deterioration such as wear accompanying the push/pull operation easily occurs.

Accordingly, an aspect of the present disclosure is to provide an elongated member for medical use that is capable of curving the distal side by the smooth push/pull operation of an operating member and favorably preventing the occurrence of deterioration due to wear that accompanies the push/pull operation or manipulation of the operating member, and that can be operated efficiently and advantageously in a pulmonary airway.

The intention of the present disclosure is achieved herein below.

In a first embodiment, an elongated member for medical use is contemplated that is used to perform a procedure in a pulmonary airway. The elongated member includes the following elements: a first member disposed on the distal side, a first lumen extending along the axial direction being formed in the first member; a second member in which a second lumen that extends along the axial direction and has a larger diameter than the first lumen is formed, the second member having a larger outer diameter than the first member and being connected to the proximal side of the first member; and an operating member that has an elongated shape and is connected to at least part of the first member, the operating member curving the first member through push/pull operation, wherein the operating member passes through the outer surface side of the first member and the inner surface side of the second member and extends along the axial direction in a substantially straight line manner.

In a second embodiment, an elongated member for medical use is provided wherein an outer groove extending along the axial direction is formed in an outer surface of the first member, the first member is connected to the second member in a state in which a proximal part of the first member is inserted into the second lumen of the second member, and the operating member passes through the outer groove to extend along the axial direction.

In another embodiment, an elongated member for medical use is provided wherein a guide member extending along the axial direction is disposed in the outer groove of the first member and the operating member passes through a lumen of the guide member to extend along the axial direction.

In an additional embodiment the elongated member for medical use is provided wherein an inner groove extending along the axial direction is formed in an inner surface of the second member and the operating member passes through the inner groove to extend along the axial direction.

In another embodiment, the instant disclosure contemplates an elongated member for medical use wherein the operating member is formed of a thin wire.

In yet another embodiment, the elongated member for medical use is provided and further includes a covering member that is so disposed as to cover an outer surface of the first member and an outer surface of the second member.

In an additional embodiment, the elongated member for medical use further includes a protective member that is so disposed as to cover the operating member.

According to the instant disclosure, the elongated member is formed by connecting the first member and the second member that are different from each other in the lumen diameter, outer diameter, and physical properties. Therefore, by selecting members suitable for the dimensions and physical properties of the respective parts of the elongated member as constituent members, the following elongated member for medical use suitable to be used in the pulmonary airway can be provided. Specifically, the distal side is given a configuration that has a small diameter and is excellent in flexibility and the proximal side is endowed with pushability (i.e., transmissibility of a pushing force). Moreover, the lumen diameter on the proximal side is set large. In the operating member to curve the first member of the elongated member passes through the outer surface side of the first member and the inner surface side of the second member and extends along the axial direction in a substantially straight line manner. Thus, the first member can be curved by a smooth push/pull operation of the operating member and deterioration, such as wear that accompanies the push/pull operation of the operating member is favorably prevented.

The instant disclosure further provides an elongated member wherein at the connection part across which the proximal end of the first member and the second member are connected, the operating member passes through the outer groove formed in the outer surface of the first member to be inserted into the lumen of the second member. This allows the operating member to extend along the axial direction in a substantially straight line manner without interfering with the second member.

In accordance with the instant disclosure, the operating member passes through the lumen of the guide member disposed in the outer groove of the first member to extend along the axial direction. Therefore, push/pull operation of the operating member can be carried out more smoothly and the occurrence of deterioration due to wear can be prevented more favorably.

According to the instant disclosure, the operating member passes through the inner groove formed in the inner surface of the second member to extend along the axial direction. This can prevent a problem that, when various kinds of procedure instruments or medical instruments are used, the procedure instrument or the like interferes with the

operating member in the lumen of the second member and the lowering of the operability is caused. In addition, the space of the lumen of the second member can be prevented from being narrowed along with the placing of the operating member.

Furthermore, the operating member is formed of a thin wire. Thus, reduction in the diameter of the elongated member can be achieved and a wide space can be ensured as the space of the lumen of the elongated member.

According to aspects of the instant disclosure, the respective parts of the elongated member can be protected by the covering member that is disposed so as to cover the outer surface of the first member and the outer surface of the second member. This can favorably prevent breakage of the elongated member during use. In addition, when various types of fluids flow via the elongated member, the occurrence of the leakage of the fluid from the elongated member can be favorably prevented.

According to the instant disclosure, when the protective member is disposed as to cover the outer surface of the operating member, the occurrence of deterioration due to wear can be prevented.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 is an outline perspective view showing a medical instrument according to an embodiment of the present disclosure.

FIG. 2 is a partial sectional view showing the overall configuration of the medical instrument according to the embodiment in a simplified manner.

FIG. 3 is an enlarged sectional view of the distal side of the medical instrument according to the embodiment.

FIGS. 4A and 4B are enlarged views showing sections of the respective parts of the medical instrument according to the embodiment; FIG. 4A being a sectional view along line 4A-4A in FIG. 3 and an enlarged view showing part of the section, FIG. 4B being a sectional view along line 4B-4B in FIG. 3 and an enlarged view showing part of the section;

FIGS. 5A and 5B are enlarged views showing sections of the respective parts of the medical instrument according to the embodiment; FIG. 5A being a sectional view along line 5A-5A in FIG. 3, FIG. 5B being a sectional view along line 5B-5B in FIG. 3.

FIGS. 6A and 6B illustrate a first member included in an elongated member according to the embodiment; FIG. 6A being an enlarged view showing the distal side of the first member, FIG. 6B being an enlarged diagram showing a joint groove formed in the first member.

FIG. 7 is a diagrammatic example of use of the elongated member for medical use according to the embodiment and is a diagram schematically showing a state in which the elongated member for medical use is introduced into a pulmonary airway;

FIG. 8 is a diagrammatic example of use of the elongated member for medical use according to the embodiment and is a diagram schematically showing a state in which the distal end of the elongated member for medical use is moved toward the peripheral side of the pulmonary airway from the state shown in FIG. 7.

FIG. 9 is a diagrammatic example of use of the elongated member for medical use according to the embodiment and is a diagram schematically showing a state in which the distal side of the elongated member for medical use is made to reach a respiratory bronchiole.

### DETAILED DESCRIPTION OF THE PREFERRED EMBODIMENTS

An embodiment of the present invention will be described below with reference to the drawings. The dimensional ratio of the drawings is exaggerated for convenience of description and is different from the actual ratio in some cases.

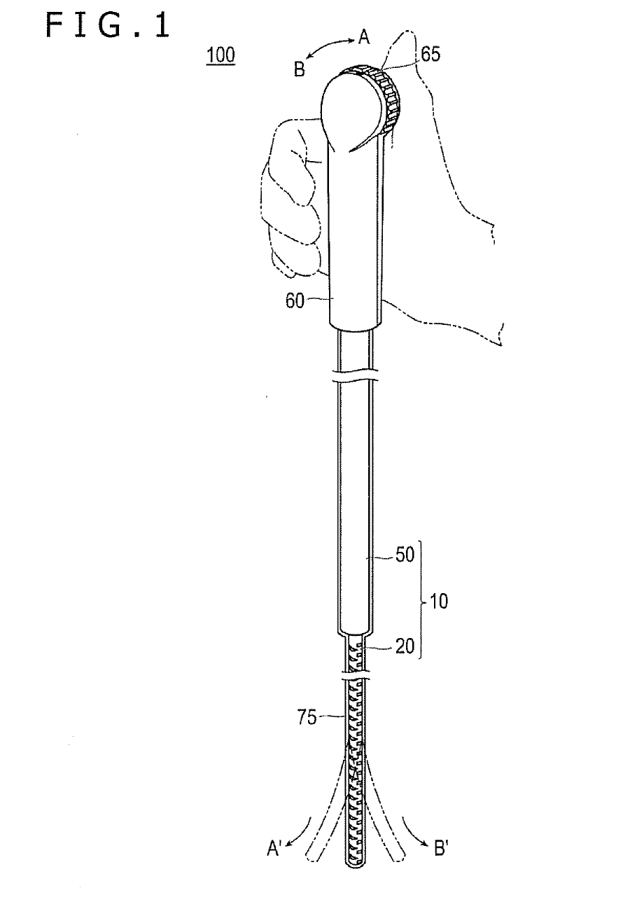

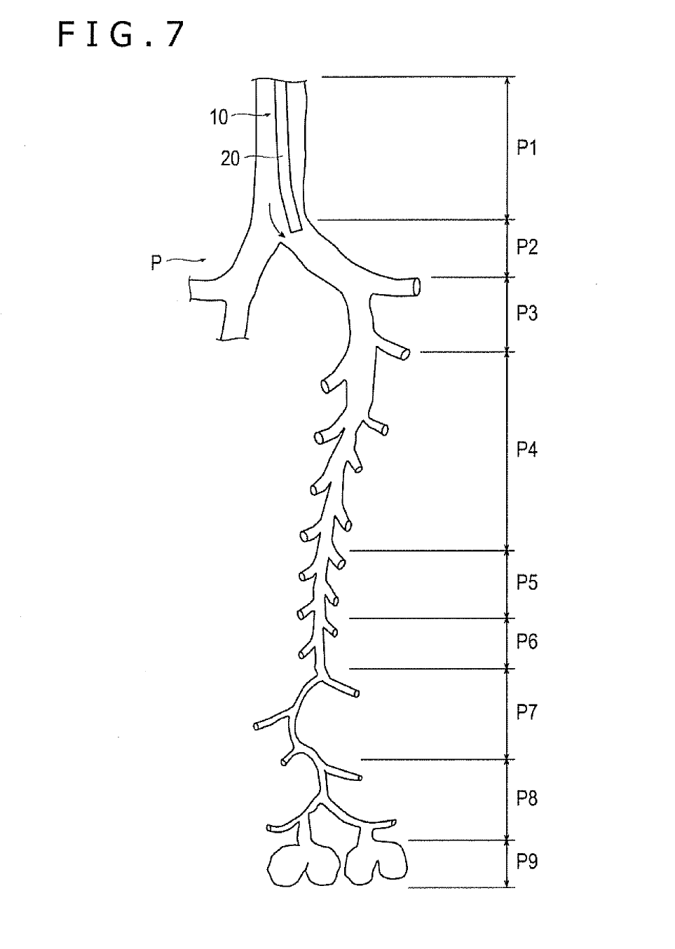

FIGS. 1 to 6B illustrate the configuration of the respective parts of an elongated member for medical use according to the embodiment. FIGS. 7 to 9 explain an example of use of the elongated member for medical use according to the embodiment.

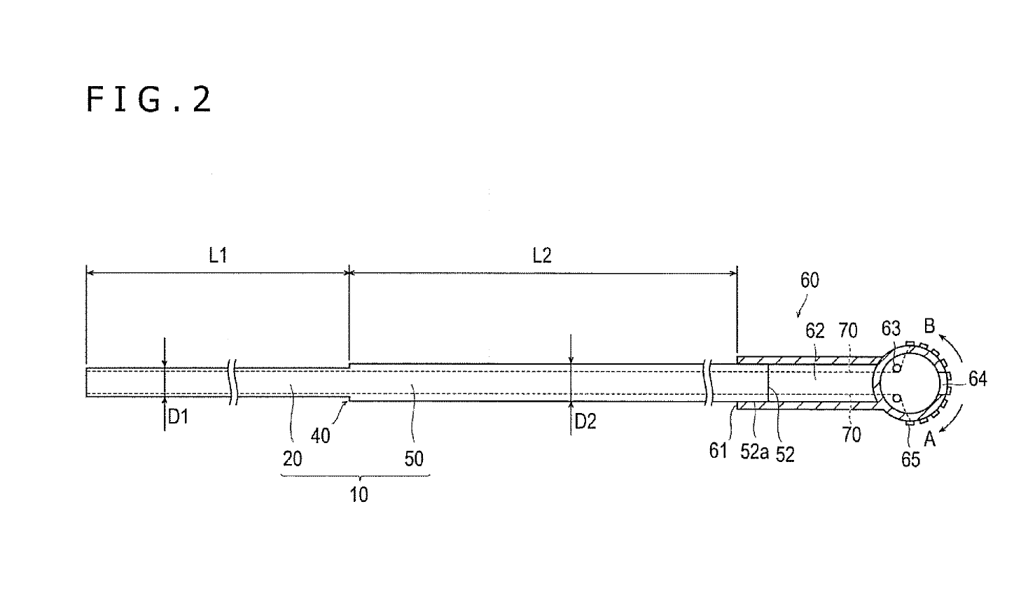

In the following description, the lower side in FIG. 1 and the left side in FIG. 2 will be referred to as the “distal side of the elongated member.” The upper side in FIG. 1 and the right side in FIG. 2 will be referred to as the “proximal side of the elongated member.” The vertical direction in FIG. 1 and the horizontal direction in FIG. 2 will be referred to as the “axial direction of the elongated member.”

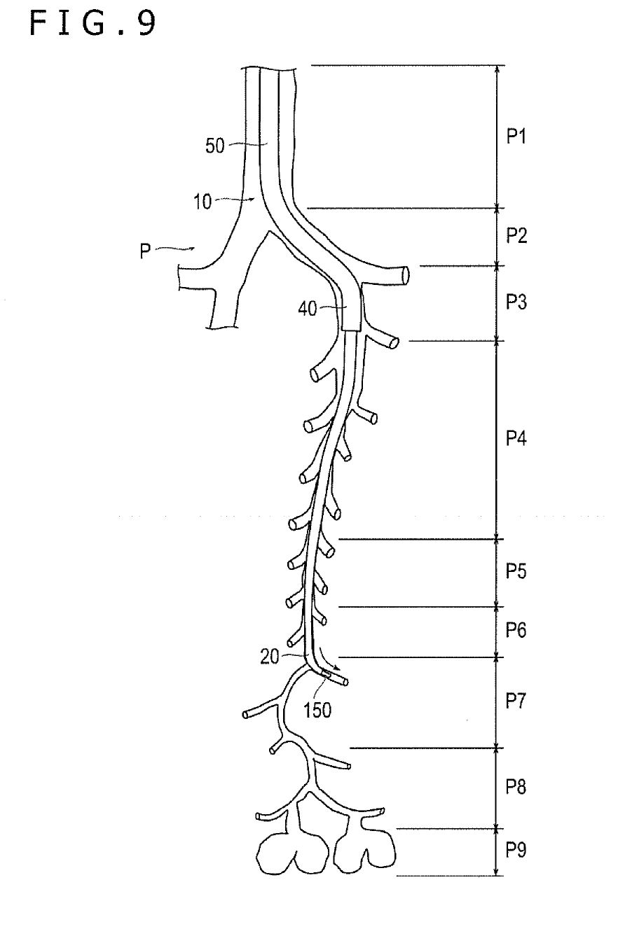

Referring to FIG. 1, an elongated member 10 for medical use according to the present embodiment (hereinafter, referred to simply as the “elongated member”) has an outer shape extended along the axial direction and its distal side is formed as a member that has the ability to curve. The elongated member 10 can be used to perform various types of procedures in a pulmonary airway P. For example, it can be used for delivery of an imaging device such as an endoscope, delivery of a biopsy instrument for obtaining a biological tissue, and delivery of a medication and suction of a body fluid, such as mucus, and the like, by use of this elongated member 10. Furthermore, it is also possible for the elongated member 10 to be used as a constituent member or the like that forms various kinds of access devices, the insertion part of a flexible endoscope, or the shaft part of a balloon catheter or the like. In the description of the present embodiment, the elongated member 10 will be explained through an example in which it is applied to a medical instrument 100 (access device) used to guide a predetermined procedure instrument 150 for medical use to a desired position in the pulmonary airway P as shown in FIG. 9.

The overall configuration of the elongated member according to the present embodiment will first be described.

As shown in FIGS. 1, 2, and 3, the elongated member 10 has a first member 20 disposed on the distal side and a second member 50 connected to a proximal part 22a of the first member 20.

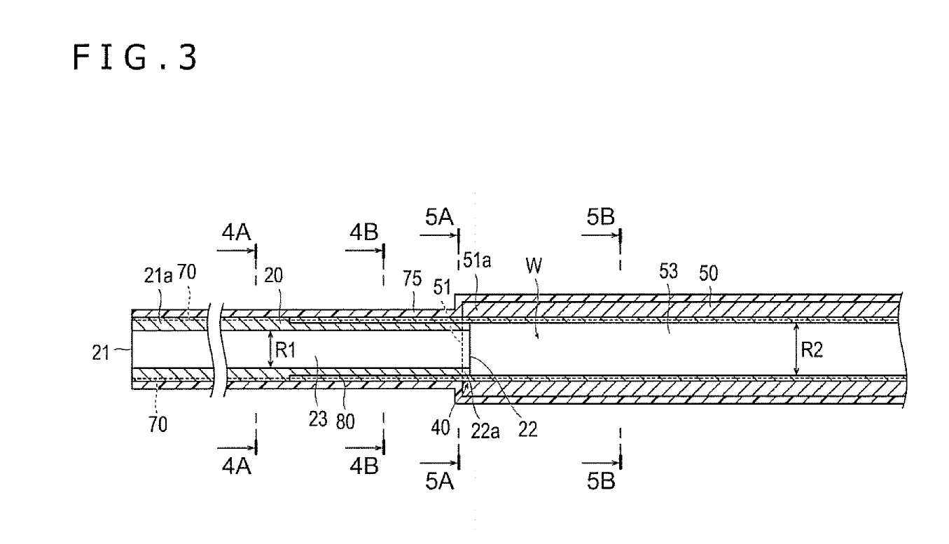

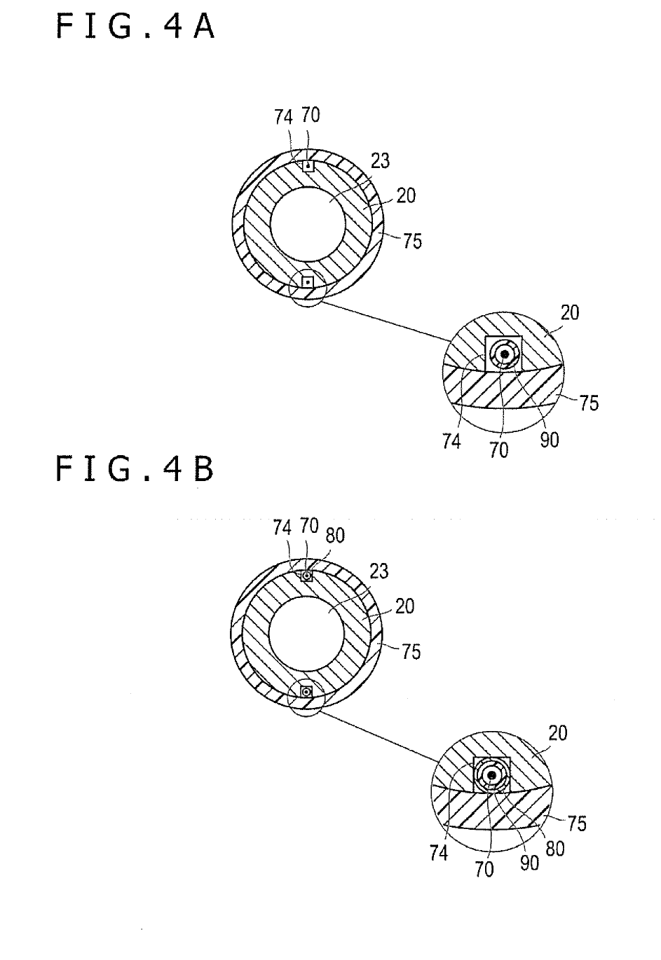

As shown in FIGS. 3, 4A, and 4B, in the first member 20, a lumen (equivalent to the first lumen) 23 extending along the axial direction is formed.

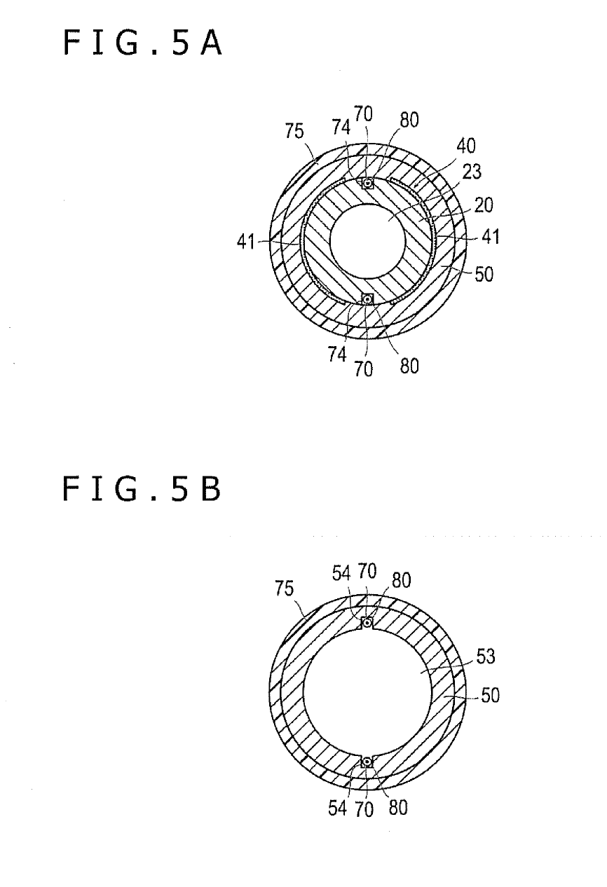

Furthermore, as shown in FIGS. 3 and 5B, in the second member 50, a lumen (equivalent to the second lumen) 53 that extends along the axial direction and has a larger diameter than the lumen 23 of the first member 20 is formed. The second member 50 is formed having a larger outer diameter than the first member 20.

As recited above, the first member 20 and the second member 50 are formed of hollow members in which the lumens 23 and 53 extend along the axial direction.

As shown in FIGS. 2 and 3, in the elongated member 10, elongated-shaped operating members 70 are provided that are connected to at least part of the first member 20 and curve the first member 20 by push/pull operation. The operating members 70 pass through the outer surface side of the first member 20 (see FIGS. 4A and 4B) and the inner surface side of the second member 50 (see FIG. 5B) and extends along the axial direction in a substantially straight line manner. The expression “extend in a substantially straight line manner” in the present specification means that an intentionally curved or bent part is not made in the operating members 70 and the operating members 70 form a straight line shape substantially in a state in which operation of curving the first member 20 is not being carried out, i.e. a state in which push/pull operation of the operating members 70 is not being carried out.

As shown in FIGS. 1 and 2, a hand operating part 60 used when pushing or pulling the operating members 70 is provided on the proximal side of the second member 50 included in the elongated member 10. In the present embodiment, the first member 20 is so formed as to curve into directions intersecting the axial direction through predetermined operation at the hand operating part 60 (A′ and B′ in FIG. 1 indicate the curving action). The hand operating part 60 and the elongated member 10 form the medical instrument 100.

Referring to FIGS. 2 and 3, the first member 20 included in the elongated member 10 is provided in order to enable settlement of the traveling direction of the elongated member 10 at each bifurcation of the pulmonary airway P. As shown in FIG. 7, in the pulmonary airway P of a living body, plural bifurcations exist from the central side (upper side in the diagram) to the peripheral side (lower side in the diagram). In performing various procedures using the elongated member 10, operation of moving the distal end of the elongated member 10 toward a desired position in the pulmonary airway P is carried out. At this time, selecting an arbitrary course at each bifurcation is enabled by appropriately curving the first member 20. In the following description, the first member 20 will be referred to as the “curving portion 20” for convenience.

Referring to FIGS. 2 and 3, the second member 50 included in the elongated member 10 is extended with a predetermined length along the axial direction. The second member 50 is formed with a length that allows the curving portion 20 disposed on its distal side to reach a desired position in the pulmonary airway P (see FIG. 9). In the following description, the second member 50 will be referred to as the “main body portion 50” for convenience.

The structure of the pulmonary airway of a living body will be described below.

Referring to FIG. 7, in the pulmonary airway P, the respective areas on the central side and the peripheral side are given names different from each other. The names of the respective areas are as follows from the central side: trachea P1, main bronchus P2, lobar bronchus P3,

bronchus P4, bronchiole P5, terminal bronchiole P6, respiratory bronchiole P7, alveolar duct PB, and alveolar sac P9. The diameters (sectional areas) of the respective areas P1 to P9 are different as shown in the diagram and the diameter becomes smaller toward the peripheral side.

It is preferable that the curving portion 20 forming the distal part of the elongated member 10 is endowed with flexibility. Furthermore, as the curving portion 20 is fabricated of a component having an outer diameter equivalent to or smaller than the diameter of a target area in which a procedure-target site exists. The purpose thereof is to allow the distal side of the elongated member 10 to easily reach the procedure-target site when various kinds of procedures for the pulmonary airway P are performed by using the elongated member 10. Meanwhile, it is preferable that the main body portion 50 disposed on the proximal side of the elongated member 10 be endowed with pushability to transmit a force that pushes the elongated member 10 into the pulmonary airway P to the distal side.

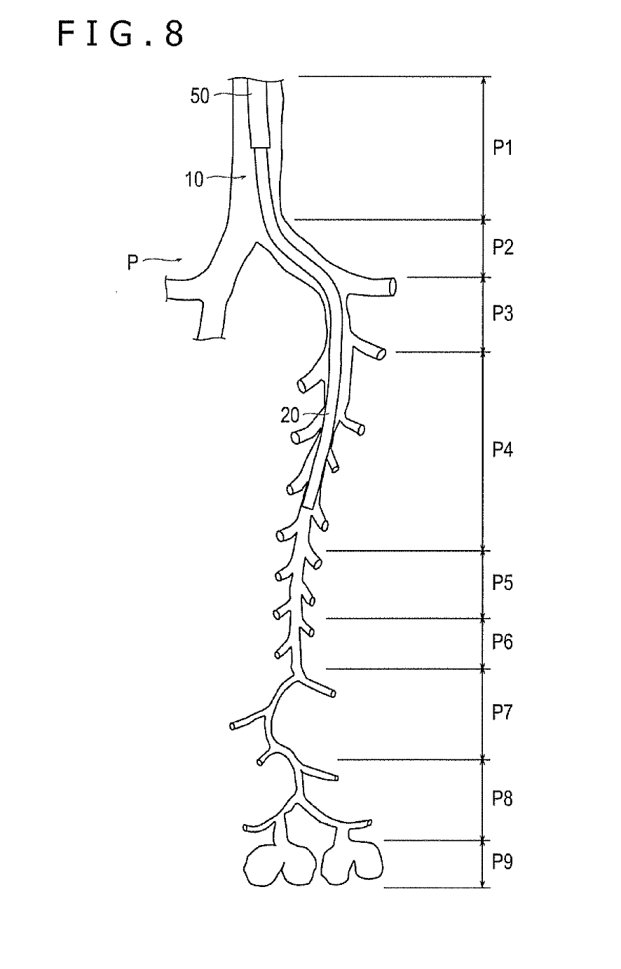

As shown in FIGS. 8 and 9, when moving the elongated member 10 in an area on the peripheral side of the pulmonary airway P, the distal side of the elongated member 10 can be moved toward the peripheral side in such a manner as to follow the pulmonary airway P by carrying out operation in a state in which the outer surfaces of the respective parts of the elongated member 10 abut against the inner wall of the pulmonary airway P. If the proximal side is endowed with sufficient pushability, the pushing force can be sufficiently transmitted from the proximal side to the distal side when the above-described operation is carried out. This allows the distal end of the elongated member 10 to easily reach the target area located on the peripheral side of the pulmonary airway P.

In the present embodiment, the periphery of the bronchiole P5 (peripheral airway), which is considered to be deeply tied to the occurrence of a lesion such as a COPD, and an area on the peripheral side, such as the respiratory bronchiole P7 close to the alveolar ducts PB and the alveolar sacs P9, are deemed as the target area, and the dimensions and physical properties of the respective parts of the elongated member 10 are so set that various kinds of procedures can be smoothly performed in this target area.

The configurations of the respective parts of the elongated member will now be described.

As shown in FIG. 3, the curving portion 20 of the elongated member 10 is formed of a member in which a distal part 21a at which a distal opening 21 is formed, the proximal part 22a at which a proximal opening 22 is formed, and the lumen 23 that communicates with the distal opening 21 and the proximal opening 22 are formed.

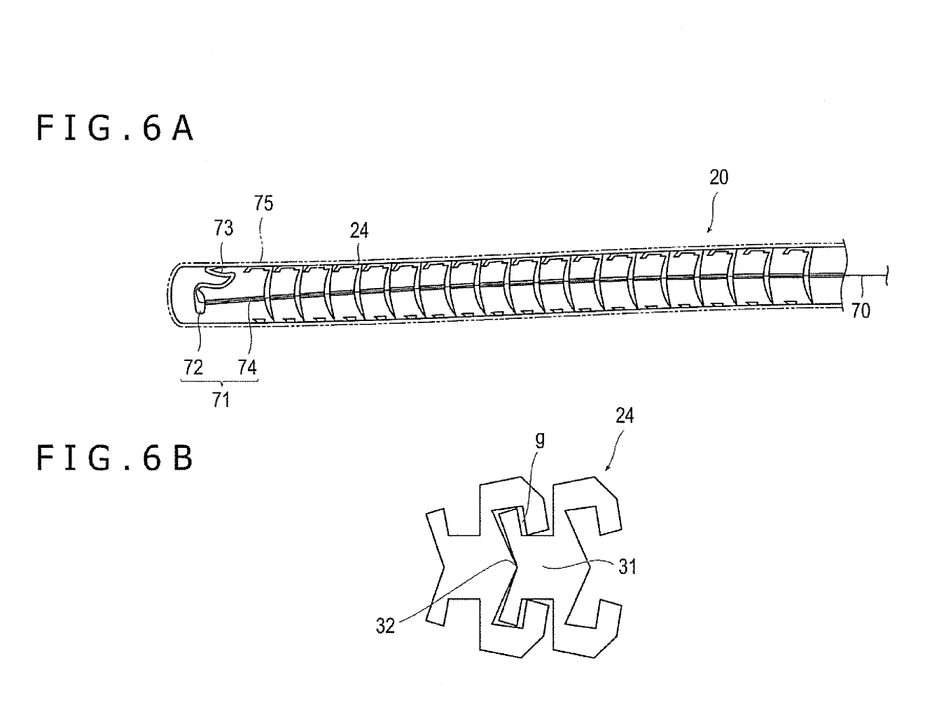

The curving portion 20 can be formed of, for example but not limited to, a hollow member that has flexibility and is so formed as to effect a curving action in accordance with push/pull operation of the operating members 70. In the present embodiment, the curving portion 20 is formed of a tubular member that is so formed as to be capable of curving through forming of joint grooves 24 to be described later (see FIG. 6A).

The tubular member forming the curving portion 20, may be fabricated from, for example but not limited to, a member formed of a metal material or a hard resin material. The metal material may be, for example, stainless steel or nickel titanium alloy. For the resin material, the following materials can be used: PP (polypropylene), HDPE (high density polyethylene), rigid polyester such as PET (polyethylene terephthalate) and PBT (polybutylene terephthalate), rigid urethane, PI (polyimide), polystyrene, PEEK (polyether ether ketone), polyamide, polyetherimide, polyamide imide, modified polyphenylene ether, and polycarbonate.

As shown in FIG. 6A, the joint grooves 24 formed in the curving portion 20 are formed by making slits (cuts) having a predetermined shape in the outer surface (or inner surface) of the tubular member. The processing method for making the slits can be selected according to the characteristics of the material used as the curving portion 20 and is not particularly limited. For example, the slits can be formed by known methods such as laser processing or etching processing can be selected.

As shown in FIG. 6A, the operating member 70 can be attached to an attachment part 71 formed in the outer surface of the curving portion 20. The attachment part 71 can be formed of, for example, a groove-shaped fixing part 72 disposed on the distal side of the curving portion 20 and an insertion groove 74 (equivalent to the “outer groove”) extended from the distal side of the curving portion 20 to the proximal side.

The operating member 70 is formed of a thin wire that is push/pull-operated along the longitudinal direction of the elongated member 10 to thereby deform the curving portion 20 into a curved or straight line shape. The material of which the wire is fabricated is not particularly limited and wires made of a metal or a resin can be used. Furthermore, the operating member 70 is not particularly limited as long as it is a component that can transmit a predetermined force from the proximal side to the distal side by push/pull operation. It is also possible that the operating member 70 is formed of a string-shaped member or a plate material having flexibility as a component other than the wire.

As shown in FIG. 6A, the operating member 70 can be attached to the curving portion 20 by fixing the operating member 70 in the fixing part 72 and fixing part of the operating member 70 in the insertion groove 74. By fixing the operating member 70 in this manner, the curving action of the curving portion 20 can be started through pulling the operating member 70. Furthermore, by loosening the pulling force, the curving action can be released and the curving portion 20 can be so deformed as to form a substantially straight line shape.

As shown in FIGS. 4A and 4B, for the operating members 70, protective members 90 may be provided and are so disposed as to cover these operating members 70. By providing the protective members 90, the influence of friction occurring when the operating members 70 are operated is reduced and the occurrence of deterioration due to wear is prevented. The protective member 90 may be fabricated of the same material as that of a covering member 75 (described herein below). The protective member 90 can be formed of a hollow member in which a lumen extends along the axial direction. In the drawings other than partial enlarged views shown in FIGS. 4A and 4B, diagrammatic representation of the protective members 90 is omitted.

As shown in FIG. 3, in the present embodiment, guide members 80 extending to range in the curving portion 20 and the main body portion 50 are provided in order to allow smooth push/pull operation of the operating members 70 and prevent the occurrence of a break such as wear due to the push/pull operation repeatedly carried out.

The guide member 80 can be formed of, for example, a thin, hollow member into which the operating member 70 is insertable. As shown in FIG. 4B, the operating members 70 pass through the lumens of the guide members 80 to extend along the axial direction. The guide member 80 may be formed from the same material as that to form the curving portion 20.

As shown in FIGS. 3, 4A, and 4B, the distal side of the guide members 80 can be disposed in the insertion grooves 74 formed in the curving portion 20. Furthermore, as shown in FIGS. 3 and 5B, the proximal side of the guide members 80 can be disposed in inner grooves 54 formed in the inner surface of the lumen 53 of the main body portion 50 for example. As shown in FIG. 3, part of the operating member 70 on the distal side is led out from the guide member 80 and this led-out part is fixed to the fixing part 72 formed in the curving portion 20 (see FIG. 6A).

In FIG. 6A, the insertion grooves 74 formed in the curving portion 20 can be formed at positions different from the positions at which the joint grooves 24 are formed in the elongated member 10 for example. Further, as illustrated in FIG. 6A the insertion grooves 74 are disposed at positions offset by 90° in the circumferential direction from the positions at which the joint grooves 24 are formed (insertion grooves 74 are disposed at positions offset from each other by 180° in the circumferential direction). Disposing them in this manner prevents the mutual interference of the respective joint grooves 24 and the operating member 70.

As shown in FIG. 6A, in the curving portion 20, a fixation assisting part 73 for fixing the operating member 70 to this curving portion 20 more strongly can be provided. The fixation assisting part 73 can be formed of a groove that is so formed as to extend with a predetermined length from the fixing part 72 along the circumferential direction of the curving portion 20. By carrying out the fixing in a state in which the operating member 70 is so positioned as to be locked to the fixation assisting part 73, the fixing strength can be enhanced.

With regard to the method for fixing the operating member 70 to the curving portion 20, an arbitrary method can be selected according to the materials of the curving portion 20 and the operating member 70. As recited herein throughout, the method of fixing can be effected by, for example, an adhesive made of a resin or a by heat fusion or the like. The instant disclosure and embodiments recited herein are not limited in the method of fixing or the substance/material used for fixing and those of skill in the art will select the appropriate fixing materials accordingly.

According to the number of insertion grooves 74 formed in the elongated member 10, two operating members 70 can be attached at positions offset from each other by 180° in the circumferential direction. As shown in FIG. 2, the proximal part of each operating member 70 is connected to a rotating member 65 provided at the hand operating part 60. The rotating member 65 is rotatably supported by a rotating shaft (not shown) provided at the hand operating part 60. When the rotating member 65 is rotated in a direction of arrow A, the curving portion 20 curves into one direction (direction of the arrow A′ in FIG. 1). When the rotating member 65 is rotated in a direction of arrow B, the curving portion 20 curves into another direction (direction of the arrow B′ in FIG. 1).

As shown in FIGS. 1 and 3, in the elongated member 10, a covering member 75 that is so disposed as to cover the outer surface of the curving portion 20 and the outer surface of the main body portion 50 can be provided. Covering member 75 can protect the respective parts of the elongated member 10. Furthermore, when causing various kinds of fluids to flow by using the elongated member 10, the occurrence of leakage of the fluid from the elongated member 10 can be favorably prevented. In addition, the efficiency of suction of a body fluid, secretion, and the like from the distal opening 21 of the curving portion 20 can also be enhanced. Moreover, it also becomes possible to prevent separation of the operating member 70 from the insertion groove 74 of the curving portion 20. The covering member 75 can be formed of, for example but not limited to, a hollow member in which a lumen extends along the axial direction.

The covering member 75, may be formed from material such as but not limited to the following materials can be used: polyolefin such as polyethylene (PE) and polypropylene (PP), polyester such as polyethylene terephthalate (PET), polyamide (PA), polyimide (PI), polyamide imide (PAI), silicone, polyurethane (PU), ethylene-vinyl acetate copolymer (EVA), polyvinyl chloride (PVC), fluorine-based resin such as polytetrafluoroethylene (PTFE), polyvinylidene fluoride (PVDF), and perfluoroalkoxy fluorine resin (PFA), and thermoplastic resin such as thermoplastic elastomer.

FIG. 6B is an enlarged diagram showing part of the joint groove 24 forming the curving mechanism. In joint groove 24, a predetermined gap g and a guide part 31 engaged with this gap g are formed. When the operating member 70 is operated, the guide part 31 moves in the gap g. This movement is transmitted along the longitudinal direction of the curving portion 20 and thereby the whole of the curving portion 20 curves. Providing a support point 32 that pivotably supports the guide part 31 as shown in FIG. 6B makes it possible to favorably transmit the movement of the guide part 31 along the longitudinal direction.

As shown in FIG. 3, the curving portion 20 and the main body portion 50 are connected in a state in which the proximal part 22a of the curving portion 20 is inserted into the lumen 53 of the main body portion 50. Specifically, the proximal part 22a of the curving portion 20 is inserted into the lumen 53 of the main body portion 50 via a distal opening 51 of the main body portion 50 and is fitted to a distal part 51a of the main body portion 50 along with the insertion. By this fitting, the curving portion 20 and the main body portion 50 are mutually connected. As shown in FIG. 5A, for example, it is also possible to apply an adhesive 41 between the curving portion 20 and the main body portion 50 or fix the curving portion 20 and the main body portion 50 by fusion bonding or welding in order to enhance the fixing strength between the curving portion 20 and the main body portion 50.

As described above, the insertion grooves 74 extending along the axial direction are formed in the outer surface of the curving portion 20. The operating members 70 pass through these insertion grooves 74 to extend along the axial direction. Furthermore, as shown in FIG. 5A, at a connection part 40 across which the curving portion 20 and the main body portion 50 are connected, the operating members 70 are so disposed as to be housed inside the insertion grooves 74. Therefore, the operating members 70 pass through the connection part 40 to be introduced into the lumen 53 of the main body portion 50 without interfering with the inner surface of the main body portion 50.

As shown in FIGS. 2 and 3, in the main body portion 50, the distal part 51a, at which the distal opening 51 is formed, a proximal part 52a at which a proximal opening 52 is formed, and the lumen 53 communicating with the distal opening 51 and the proximal opening 52 are formed. In FIG. 3, the distal opening 51 of the main body portion 50 is virtually indicated by a dashed line.

As described above, the proximal part 22a of the curving portion 20 is inserted into the distal side of the lumen 53 of the main body portion 50. Furthermore, as shown in FIG. 2, the proximal part 52a of the main body portion 50 is inserted into a distal opening 61 formed in the hand operating part 60 and is fitted thereto along with the insertion. The main body portion 50 is fixed to the hand operating part 60 by this fitting.

As shown in FIGS. 3 and 5B, the guide members 80 extending from the distal side of the elongated member 10 to the proximal side can be disposed in the inner grooves 54 formed in the inner surface of the main body portion 50 for example. The operating members 70 inserted into the guide members 80 pass through the inner grooves 54 together with the guide members 80 to extend along the axial direction. Disposing the guide members 80 in the inner grooves 54 prevents narrowing of the usable space in the lumen 53 of the main body portion 50 due to the placing of the guide members 80.

The material forming the main body portion 50 is not particularly limited as long as it is endowed with pushability that allows transmission of a pushing force to the side of the curving portion 20 disposed on the distal side. For example, main body portion 50 may be formed of a material selected from: polyvinyl chloride, polyethylene, polypropylene, cyclic polyolefin, polystyrene, poly(4-methylpentene-1), polycarbonate, acrylic resin, acrylonitrile-butadiene-styrene copolymer, polyester such as polyethylene terephthalate and polyethylene naphthalate, butadiene-styrene copolymer, polyamide (e.g. nylon 6, nylon 6.6, nylon 6.10, nylon 12), and various kinds rubber materials such as natural rubber, butyl rubber, isoprene rubber, butadiene rubber, styrene-butadiene rubber, and silicone rubber. In one example the main body portion 50 is formed of a tubular member composed of a spiral tube formed by winding a strip plate into a spiral shape, a mesh net covering the spiral tube, and a resin outer coat covering the outer circumferential surface of the net. Furthermore, in another embodiment, it is also possible that the main body portion 50 may formed of a tubular member having a layered structure formed by covering a hollow member formed of a metal coil by a metal blade. Moreover, it is also possible to use a tubular member having a layered structure formed of the same kind of material. Using the same kind of material can enhance the bonding strength between both members when both members are bonded by using an adhesive or the like.

As shown in FIG. 2, the hand operating part 60 has the distal opening 61, into which the proximal end of the main body portion 50 is inserted, a space part 62 extending along the axial direction, support parts 63 that assist the operating members 70 in push/pull operation, an insertion opening 64 formed at the proximal part, and the rotating member 65 used to carry out push/pull operation of the operating members 70.

The space part 62 of the hand operating part 60 is disposed coaxially with each of the lumen 23 of the curving portion 20 and the lumen 53 of the main body portion 50. This space part 62 and the lumens 23 and 53 of the respective portions form a working lumen w for inserting a predetermined medical device, another medical instrument, or the like into the medical instrument 100. For example, when the procedure instrument 150 for medical use is delivered into the pulmonary airway P via the medical instrument 100 (see FIG. 9), the procedure instrument 150 is inserted into the inside of the hand operating part 60 via the insertion opening 64 formed in the hand operating part 60. The procedure instrument 150 is then inserted into the working lumen w and its distal side is protruded from the distal opening 21 of the curving portion 20. The procedure instrument 150 can be thereby guided to a procedure-target site.

The rotating member 65 of the hand operating part 60 is rotatably attached in a state of being connected to the operating members 70. A concave-convex part on which a finger can be made to rest is formed on the surface of the rotating member 65. As shown in FIG. 1, for example, the curving operation of the curving portion 20 can be carried out by simple operation of rotating the rotating member 65 with use of a finger of one hand. The hand operating part 60 may be formed, for example, from a rigid plastic material or a metal material.

The dimensions and physical properties of the respective parts of the elongated member will be described.

Referring to FIG. 2 once again, it is preferable that a length L1 of the curving portion 20 in the axial direction is at least 10 mm and at most 60 mm for example. Note that the length L1 of the curving portion 20 described here does not include the length of the part inserted into the lumen 53 of the main body portion 50.

As shown in FIG. 7, when the elongated member 10 is moved toward the peripheral side, the elongated member 10 can be moved along the pulmonary airway P by moving the elongated member 10 in such a manner that it abuts against the inner wall surface of the pulmonary airway P. At the initial stage of the insertion, specifically for example while the elongated member 10 is being moved on the central side, the elongated member 10 can be smoothly moved by making the curving portion 20 of the elongated member 10 abut against (i.e., get caught on) the inner wall surface of the lobar bronchus P3 (around the second bifurcation) located on the central side. However, if the length L1 of the curving portion 20 is smaller than the diameter of the main bronchus P2 (around the first bifurcation), bending and inflection easily occur in the curving portion 20 in the midst of the movement of the curving portion 20 in the main bronchus P2 and therefore it is difficult for the curving portion 20 to reach the lobar bronchus P3. Therefore, the length L1 of the curving portion 20 is set to at least 10 mm, which is a general diameter of the main bronchus P2. To prevent the occurrence of the above-described problem, it is preferable that the length L1 of the curving portion 20 is at least 14 mm.

When pushing the elongated member 10 in order to further advance the curving portion 20 toward the peripheral side after the distal end of the curving portion 20 reaches the peripheral airway, the curving portion 20 can be moved toward the peripheral side by at most 60 mm due to the elongation of the peripheral airway. Therefore, by setting the upper limit of the length L1 of the curving portion 20 to approximately 60 mm, the curving portion 20 can be prevented from being formed unnecessarily long with enabling of inclusion of a wider area of the peripheral area in procedure-target sites. The peripheral airway is elongated by at least 40 mm. Therefore, it is more preferable that the length L1 of the curving portion 20 is at most 40 mm in terms of preventing the curving portion 20 from being formed unnecessarily long.

Referring again to FIG. 2, it is preferable that an outer diameter D1 of the curving portion 20 is at least 2.0 mm and at most 4.5 mm. As described herein, the outer diameter D1 of the curving portion 20 means an outer diameter including the thickness of the covering member 75 if the covering member 75 is used, and also means an outer diameter including the thickness of a coating material to be described herein below if the coating material is used.

The diameter of the respiratory bronchiole P7 in the pulmonary airway P is generally 0.5 to 1.0 mm (see FIG. 7). When the curving portion 20 is pushed into the respiratory bronchiole P7, the respiratory bronchiole P7 dilates to at most about 1.0 mm to 2.0 mm. Therefore, if the outer diameter D1 of the curving portion 20 is 2 mm, the curving portion 20 can be so moved as to abut against the inner wall surface of the respiratory bronchiole P7. Furthermore, referring to FIG. 3, it is preferable that a diameter R1 of the lumen 23 of the curving portion 20 is at least 1 mm so that various kinds of medical devices and medical instruments can be inserted. For this reason, in consideration of the necessary minimum wall thickness of the curving portion 20, it is preferable that the outer diameter D1 of the curving portion 20 is at least 2 mm in terms of the relationship with the diameter R1 of the lumen 23.

The diameter of the peripheral airway of the pulmonary airway P is generally 2.0 mm. When the curving portion 20 is pushed into the peripheral airway, the peripheral airway dilates to at most about 4.5 mm. Therefore, if the outer diameter D1 of the curving portion 20 is 4.5 mm, the curving portion 20 can be moved so as to abut against the inner wall surface of the peripheral airway. For this reason, it is preferable that the upper limit of the outer diameter D1 of the curving portion 20 be 4.5 mm. Because the peripheral airway dilates to only about 3.5 mm in some cases, it is more preferable that the outer diameter D1 of the curving portion 20 is at most 3.5 mm in terms of the invasiveness.

With reference to FIG. 2, it is preferable that a length L2 of the main body portion 50 along the axial direction is at least 230 mm and at most 800 mm. Note that the length of the main body portion 50 described here does not include the length of the part inserted into the space part 62 of the hand operating part 60.

Forming the main body portion 50 having the above described length L2 enables the distal end of the curving portion 20 to surely reach a site on the peripheral side relative to the bronchus P4 when the elongated member 10 is introduced into the pulmonary airway P orally or nasally. Thus, it is possible to favorably perform various procedures in the peripheral airway including the bronchiole P5 and in the respiratory bronchiole P7.

The outer diameter D2 of the main body portion 50 is advantageously in the range of from approximately 0 mm to approximately 5.0 mm. As shown in FIG. 9, when the main body portion 50 has the outer diameter D2 in this range, and when the elongated member 10 is moved in the pulmonary airway, the curving portion 20 on the peripheral side can be moved and a state is retained in which the main body portion 50 abuts against the inner wall surface of the central side. Furthermore, the pushing force given from the proximal side is favorably transmitted in the axial direction via the main body portion 50 and thus the pushability of the elongated member 10 can be enhanced. The outer diameter D2 of the main body portion 50 described here is intended to refer to an outer diameter including the thickness of the covering member 75 if such covering member 75 is used, and an outer diameter including the thickness of a coating material (described herein below) if the coating material is used.

As shown in FIG. 3, the elongated member 10 is so formed such that a diameter R2 of the lumen 53 of the main body portion 50 is larger than the diameter R1 of the lumen 23 of the curving portion 20. Therefore, when procedure instruments 150 are introduced into the pulmonary airway P by using the elongated member 10, the procedure instruments 150 can be easily moved from the side of the main body portion 50 to the side of the curving portion 20. Furthermore, when the elongated member 10 is applied to a suction device or a discharge device, the suction power can be enhanced or the discharge amount can be increased. The diameter R2 of the lumen 53 of the main body portion 50 can be in the range of from approximately 2.0 mm to approximately 4.5 mm, and the diameter R1 of the lumen 23 of the curving portion 20 can in the range of approximately 1.2 mm to approximately 3.7 mm. In the elongated member 10 according to the present embodiment, the diameter R2 of the lumen 53 of the main body portion 50 is identical to or smaller than the outer diameter D1 of the curving portion 20 so that the proximal part 22a of the curving portion 20 can be fitted to the lumen 53 of the main body portion 50.

The outer surface of the curving portion 20 can be covered by a coating material having slipperiness. Enhancing the slipperiness of the curving portion 20 can further enhance the pushability of the elongated member 10. The coating material may be, for example, a material that forms a coating film on the surface of the curving portion 20 or a material formed as a film material covering the surface of the curving portion 20. In the case of providing the covering member 75 on the curving portion 20, a configuration in which the coating material is applied on the covering member 75 can be employed. The coating material provided on covering member 75 may be a hydrophilic material or a hydrophobic material.

Examples of the hydrophilic material include cellulose-based polymer substance, polyethylene oxide-based polymer substance, maleic anhydride-based polymer substance (e.g. maleic anhydride copolymer such as methyl vinyl ether-maleic anhydride copolymer), acrylamide-based polymer substance (e.g. polyacrylamide and block copolymer of poly(glycidyl methacrylate) dimethylacrylamide (PGMA-DMAA)), water-soluble nylon, polyvinyl alcohol, and polyvinylpyrrolidone. In many cases, such a hydrophilic material exerts lubricity through moistening (water absorption) and reduces the frictional resistance (sliding resistance) against the inner wall of a wet biological organ. This enhances the slidablity and operation of the elongated member 10

Examples of the hydrophobic material include polyamide, polyimide, polyurethane, polystyrene, silicone resin, fluorine-based resin (PTFE, ETFE, etc.), and composite materials of them. When such a hydrophobic material is used, the same effects as those by the above-described hydrophilic material can be exerted.

It is also possible to employ a configuration in which the inner surface of the lumen 23 of the curving portion 20 is covered by the coating material. This configuration makes it possible to smoothly insert the procedure instrument 150 into the curving portion 20, or the withdrawal of it from the curving portion 20. Furthermore, the outer surface of the operating member 70, and the outer surface of the protective member 90 covering the operating member 70 may be covered by the coating material. This can reduce friction when using the operating member 70 and also prevents the occurrence of damage and wear.

The elongated member 10 is so formed that the torque transmissibility of the main body portion 50 is higher than that of the curving portion 20. By employing this configuration, when rotational operation or twist operation is carried out in a state in which the curving portion 20 abuts against the inner wall surface of the peripheral side of the pulmonary airway P, the action of the curving portion 20 following this operation can be suppressed. This can favorably prevent the occurrence of damage or the like on the pulmonary airway P. Meanwhile, because the torque transmissibility of the main body portion 50 is kept, the main body portion 50 can be moved with rotational operation or twist operation and thus the operability of the elongated member 10 can be enhanced.

An example of method of use of the elongated member according to the present embodiment will now be described.

Referring to FIG. 7, a part on the distal side of the medical instrument 100, specifically the distal end of the curving portion 20 of the elongated member 10, is inserted into the pulmonary airway P from the central side of the pulmonary airway P. The insertion can be carried out orally or nasally for example.

After the distal end of the curving portion 20 reaches the first bifurcation, the curving portion 20 is curved as shown in FIG. 7 to orient the distal end of the curving portion 20 toward a desired direction. Since the operating members 70 are disposed along the axial direction in a substantially straight line manner as described above, push/pull operation of the operating members 70 can be carried out smoothly.

The work of moving the elongated member 10 can be performed while the course of the elongated member 10 is appropriately decided by using an imaging unit such as an endoscope. In this manner, an image whose field of view includes the distal side of the elongated member 10 can be acquired by protruding the endoscope from the distal opening 21 of the curving portion 20 through the working lumen w formed inside the medical instrument 100. The elongated member 10 can be moved along the desired course by repeating the protrusion of the endoscope from the distal opening 21 of the curving portion 20, check of the status of the distal side of the elongated member 10, and the forward movement of the elongated member 10.

Referring to FIG. 8, the elongated member 10 is further pushed ahead toward the peripheral side of the pulmonary airway P. At this time, by the contact of the main body portion 50 of the elongated member 10 with the inner wall surface of the pulmonary airway P in the respective areas of the pulmonary airway P, the elongated member 10 can be so moved as to follow the pulmonary airway P.

Referring to FIG. 9, the elongated member 10 is pushed ahead until the curving portion 20 of the elongated member 10 reaches the respiratory bronchiole P7 located on the peripheral side. When the elongated member 10 reaches the respiratory bronchiole P7, the outer surface of the curving portion 20 abuts against the inner wall surface of the respiratory bronchiole P7.

Thus, it becomes possible to move the curving portion 20 along the inner wall surface of the respiratory bronchiole P7. While the curving portion 20 is being moved in the respiratory bronchiole P7, the outer surface of the main body portion 50 of the elongated member 10 is kept abutting against the inner wall surface of the lobar bronchus P3 for example. Working in such a status allows the pushing force to be favorably transmitted to the side of the curving portion 20 via the main body portion 50, enabling the curving portion 20 to move more smoothly.

After the distal end of the curving portion 20 reaches a desired position in the pulmonary airway P, a procedure instrument 150 is introduced via the elongated member 10. By using the procedure instrument 150, various kinds of procedures can be performed at a position close to a procedure-target site existing on the peripheral side. The procedure instrument 150 may include the following instruments: biopsy device, ultrasonic diagnosis device, microcatheter, ablation device, cryocatheter, radio-frequency ablation catheter, microwave ablation catheter, and PDT probe. When an endoscope is used in combination, interchanging the endoscope with the procedure instrument 150 is appropriately performed.

After a predetermined procedure is performed, the procedure instrument 150 is withdrawn from the medical instrument 100. After the procedure instrument 150 is withdrawn from the living body, the medical instrument 100 is withdrawn from the living body to end the manipulation. The manipulation may be continued by carrying out operation of moving the elongated member 10 again toward another procedure-target site through repetition of the same process.

As recited above, according to the present embodiment, the elongated member 10 is formed by connecting the curving portion 20 and the main body portion 50 that are different from each other in the lumen diameter, outer diameter, and physical properties. Therefore, by selecting members suitable for the dimensions and physical properties of the respective parts of the elongated member 10 as constituent members, the following elongated member 10 for medical use suitable to be used in the pulmonary airway P can be provided. Specifically, the distal side is given a configuration that has a small diameter and is excellent in flexibility and the proximal side is endowed with pushability. Moreover, the lumen diameter on the proximal side is set large. Furthermore, the operating member 70 to curve the curving portion 20 of the elongated member 10 passes through the outer surface side of the curving portion 20, the connection part 40, across which the curving portion 20 and the main body portion 50 are connected, and the inner surface side of the main body portion 50 and extends along the axial direction in a substantially straight line manner. Thus, the curving portion 20 can be curved by the smooth push/pull operation of the operating member 70 and the occurrence of deterioration, such as wear accompanying the push/pull operation of the operating member 70, can be favorably prevented.

At the connection part 40, across which the proximal part 22a of the curving portion 20 and the main body portion 50 are connected, the operating member 70 passes through the insertion groove 74 formed in the outer surface of the curving portion 20 to be inserted into the lumen 53 of the main body portion 50. This allows the operating member 70 to extend along the axial direction in a substantially straight line manner without interfering with the main body portion 50.

Additionally, the operating member 70 passes through the lumen of the guide member 80 disposed in the insertion groove 74 of the curving portion 20 to extend along the axial direction. Therefore, push/pull operation of the operating member 70 can be carried smoothly and the occurrence of deterioration due to wear can be favorably prevented.

Operating member 70 passes through the inner groove 54 formed in the inner surface of the main body portion 50 to extend along the axial direction. This can prevent the procedure instruments 150 or medical instruments from interfering with the operating member 70 in the lumen 53 of the main body portion 50. In addition, the space of the lumen 53 of the main body portion 50 can be prevented from being narrowed along with the placing of the operating member 70.

The operating member 70 is preferably formed of a thin wire. Thus, reduction in the diameter of the elongated member 10 can be achieved and a wide space can be ensured for the space of the working lumen w of the elongated member 10.

The respective parts of the elongated member 10 are protected by the covering member 75, which is so disposed as to cover the outer surface of the curving portion 20 and the outer surface of the main body portion 50. This can prevent breakage in the elongated member 10 during use. In addition, when various types of fluids flow via elongated member 10, the occurrence of leakage of the fluid from the elongated member 10 can be favorably prevented.

Moreover, the protective member 90, which is disposed as to cover the outer surface of the operating member 70. This can prevent the occurrence of deterioration due to wear accompanying push/pull operation of the operating member 70 further favorably.

The elongated member is not limited only to the above-described embodiment and can be appropriately modified based on the description of the scope of claims as long as an operating member is so configured as to pass through the outer surface side of a first member, a connection part across which the first member and a second member are connected, and the inner surface side of the second member and extend along the axial direction in a substantially straight line manner in an elongated member, and wherein the first member is connected to the second member, and an operating member that is push/pull-operated to curve the first member.

For example, the configuration of the curving portion 20 provided in the elongated member 10 is not limited to one in which a curving action is made based on the curving mechanism formed with the joint grooves 24, and in the alternative may be one formed by juxtaposing tubular members such as joint rings in the longitudinal direction. The configuration and of the hand operating part 60 can also be changed as long as it can curve the curving portion 20. It is also possible to use the elongated member 10 alone as a medical instrument without providing the hand operating part 60. The procedure instruments 150 for medical use and the contents of procedures that are exemplified are only examples and it is possible to perform various procedures with use of a procedure instruments other than those exemplified herein.

The elongated member is not limited to a configuration in which two members are connected and it is also possible to form it by connecting three or more members for example.

For example, at the distal side of the elongated member 10, a balloon can be disposed which is so formed as to be capable of inflation and deflation through injection and discharge of a fluid. By inflating the balloon during administration of a medication or the like via the elongated member 10, the administration can be performed in a state in which the position of the elongated member 10 is fixed and settled. Thus, the medication administration can be efficiently performed.

Furthermore, it becomes possible to occlude the peripheral side of the pulmonary airway P by the balloon and administer medication or the like to the distal side relative to the occluded part. Therefore, local administration of the medication is also enabled. When using the balloon, the balloon may be so disposed as to cover the covering member 75 or may be disposed directly on the outer surface of the elongated member 10. The balloon is connected to a fluid introduction lumen in such a manner that a fluid can be introduced to the balloon from the hand side, and the balloon is inflated by introduction of a fluid from this fluid introduction lumen. The fluid introduction lumen may be disposed inside or outside the elongated member 10. The balloon is formed of a material that expands and contracts, such as silicone. As a result, the balloon is brought into close contact with a biological lumen when being inflated and operates in a state in which the elongated member 10 is fixed at a predetermined position. The balloon is not, however, limited to silicone and a material that does not expand and contract, such as nylon or polyethylene, may be disposed in a folded state. If the balloon is formed of such a material, dilation of a narrowed part generated in a bronchus is also enabled.

While illustrative and presently preferred embodiments of the present invention have been described in detail herein, it is to be understood that the inventive concepts may be otherwise variously embodied and employed without departing from the spirit and scope of the invention. The appended claims are intended to be construed to include such variations and equivalents.

What is claimed is:

1. An elongated member for medical use that is used to perform a procedure in a pulmonary airway, the elongated member comprising:
a first member disposed on a distal side, the first member having a first lumen formed in the first member, the first lumen extending along an axial direction in the first member;
a second member having a second lumen that extends along the axial direction, the second lumen having a diameter that is larger than the first lumen, the second member having a larger outer diameter than the first member and being connected to a proximal side of the first member; and
an operating member having an elongated shape, the operating member connected to at least part of the first member, the operating member curving the first member through push/pull manipulation,
wherein the operating member passes through an outer surface side of the first member and an inner surface side of the second member and extends along the axial direction in a substantially straight line manner.

2. The elongated member according to claim 1,
wherein an outer groove extending along the axial direction is formed in an outer surface of the first member,
and wherein the first member is connected to the second member in a manner in which a proximal part of the first member is inserted into the second lumen of the second member, and
the operating member passes through the outer groove to extend along the axial direction.

3. The elongated member according to claim 2,
wherein a guide member extending along the axial direction is disposed in the outer groove of the first member, and
the operating member passes through a lumen of the guide member to extend along the axial direction.

4. The elongated member according to claim 1,
wherein an inner groove extending along the axial direction is formed in an inner surface of the second member, and
the operating member passes through the inner groove to extend along the axial direction.

5. The elongated member for medical use according to claim 1, wherein the operating member is formed of a thin wire.

6. The elongated member according to claim 1, further comprising a covering member that is so disposed as to cover an outer surface of the first member and an outer surface of the second member.

7. The elongated member according to claim 1, further comprising a protective member that is so disposed as to cover the operating member.
